(19) United States
(12) Patent Application Publication
(10) Pub. No.: US 2007/0123725 A1
(43) Pub. Date: May 31, 2007

(54) Process for the production of polyols based on natural oils

(75) Inventor: Klaus Lorenz, Dormagen (DE)

Correspondence Address: BAYER MATERIAL SCIENCE LLC, 100 BAYER ROAD, PITTSBURGH, PA, 15205, US

(73) Assignee: Bayer MaterialScience AG

(21) Appl. No.: 11/602,787

(22) Filed: Nov. 21, 2006

(30) Foreign Application Priority Data
Nov. 26, 2005 (DE) 102005056432.1

(51) Int. Cl. C07C 51/16; C07C 51/34

(52) U.S. Cl. 554/138

(57) ABSTRACT

The present invention relates to a process for the production of polyols based on natural unsaturated oils.

### FIELD OF THE INVENTION

The present invention relates to a process for the production of polyols based on natural, unsaturated oils.

### BACKGROUND OF THE INVENTION

Polyols based on sustainable raw materials such as fatty acid triglycerides, sugars, glycerol and dimer fatty alcohols are already widely used as a raw material in the production of polyurethane materials. The use of such components will further increase in future, because products from renewable sources are given a positive rating in environmental audits and the availability of raw materials based on petrochemicals will decline over the long term. Fatty acid triglycerides are largely obtained from sustainable sources and are therefore a cost-effective basis for polyurethane raw materials among other things. A disadvantage is the lack of fatty acid triglycerides already incorporating the reactive hydrogen atoms which are necessary for reaction with isocyanates. The main exception in this connection is castor oil, whilst the rare lesquerella oil also contains hydroxyl groups. The availability of castor oil is restricted, however, due to its geographically limited areas of cultivation.

The reactive hydrogen atoms that are necessary for use in the polyurethane industry therefore have to be introduced into the vast majority of naturally occurring oils by means of chemical processes. According to the state of the art, methods for achieving this substantially make use of the double bonds occurring in the fatty acid esters of many oils, whilst methods for releasing glycerol from triglycerides are also described. Reactions with alkylene oxides are also described as a means of modifying these polyols in terms of molecular weight and interfacial surface tension, increasing their compatibility with conventional polyethers and bringing their processing characteristics into line with those of conventional polyols. For example:

US-A 2002/0058774 describes the production of polyfunctional polyols based on unmodified or blown oils. The products are obtained in a two-stage interesterification process from the oil and polyfunctional alcohols and saccharides. An acid-catalyzed reaction is not described in US-A 2002/0058774.

US-A 2003/0191274 claims reaction products obtained in a two-stage process from unmodified or blown oils and sugars together with glycerol as starter components for alkoxylation reactions. Acid-catalyzed epoxidations and epoxide ring openings are not described in US-A 2003/0191274.

WO-A 2004020497 claims the use of double metal cyanide complex (DMC) catalysts in the production of alkylene oxide adducts based on starter components from sustainable sources. Castor oil is used as the preferred starter component, and oils subsequently modified with hydroxyl groups can also be used. The specification gives no information about how such hydroxyl group-modified oils that are suitable for alkoxylation can be obtained in situ by simple means.

U.S. Pat. No. 6,686,435 claims the reaction of epoxidized oils with alcohol/water mixtures catalyzed by HBF₄ for the purposes of ring opening and obtaining hydroxyl groups. The epoxidation reaction can also directly precede the ring-opening reaction. A subsequent reaction of these products with alkylene oxides to form polyether polyols and the acid-catalyzed epoxidation are not described in U.S. Pat. No. 6,686,435.

EP-A 1112243 describes the production of supported DMC catalysts which are suitable for the alkoxylation of fatty alcohols and which can be separated on completion of the reaction of the starter with the alkylene oxide. The production of the fatty alcohols is not disclosed in EP-A 1112243.

EP-A 0259722 deals with the acid-catalyzed ring opening of oxirane rings on epoxidized fatty acid triglycerides with low-molecular-weight monofunctional alcohols. Polyols obtained in such a way can optionally undergo further alkoxylation, although no details are given of suitable reaction conditions.

JP-A 05163342 claims the alkoxylation of castor oil or modified castor oil in the presence of DMC catalysts. How these modified castor oils are obtained is not described in JP-A 05163342.

DE-A 3323880 explains how castor oil or modified castor oil can be reacted with alkylene oxides, catalyzed by Lewis acids or double metal cyanide complexes, to form polyether polyols. The preparation of modified castor oils is not described, however.

U.S. Pat. No. 6,548,609 describes Lewis acids such as tetrabutyl titanate or triphenyl phosphite as catalysts for the reaction of epoxidized oils with alcohols, amines or alkanolamines. The further reaction of these products with alkylene oxides is not described.

DE-A 19812174 describes interesterification products obtained from fatty acid triglycerides and polyhydric alcohols, which are subsequently alkoxylated. Acid-catalyzed epoxidations and epoxide ring openings are not described in DE-A 19812174.

U.S. Pat. No. 6,420,443 claims alkylene oxide adducts obtained from fatty acid triglycerides as compatibility agents in hydrocarbon-blown rigid foam formulations. Alkylene oxide adducts of this type also include, in addition to reaction products from castor oil and alkylene oxides, those from ring-opened epoxidized fatty acid triglycerides and alkylene oxides. Simple reaction pathways from the unmodified fatty acid triglycerides via their in-situ epoxidation/ring opening to the corresponding alkylene oxide addition products are not shown.

DE-A 19545165 describes the use of ring-opening products of epoxidized triglycerides as a cell-opening component in rigid and flexible polyurethane foam formulations. No details are given in DE-A 19545165 of processes for obtaining these ring-opening products or of polyols obtainable from them after alkoxylation.

WO-A 2004096883 describes how higher-functional polyesters, which are used as a polyol component in flexible foam formulations, can be obtained from methylol group-containing derivatives produced by hydroformylation/hydrogenation from fatty acids or fatty acid esters, by means of esterification or interesterification in the presence of polyfunctional polyols. The epoxidation of natural oils is not described in WO-A 2004096883.

DE-A 4332292 describes the oxidation of triglycerides in the presence of hydrogen peroxide and carboxylic acids. A process for how alkylene oxide-reacted polyether polyols can be produced from the triglycerides is not disclosed in DE-A 4332292.

### SUMMARY OF THE INVENTION

The present invention therefore provides a simple process allowing unmodified unsaturated fatty acid triglycerides to be converted into alkylene oxide adducts having a wide OH value range which are compatible with conventional polyethers obtained on the basis of petrochemical intermediates. These and other advantages and benefits of the present invention will be apparent from the Detailed Description of the Invention herein below.

### DETAILED DESCRIPTION OF THE INVENTION

The present invention will now be described for purposes of illustration and not limitation. Except in the operating examples, or where otherwise indicated, all numbers expressing quantities, percentages, OH numbers, functionalities and so forth in the specification are to be understood as being modified in all instances by the term “about.” Equivalent weights and molecular weights given herein in

Daltons (Da) are number average equivalent weights and number average molecular weights respectively, unless indicated otherwise.

The present invention provides a process for the production of polyols based on natural oils involving
a) oxidizing unsaturated triglycerides with peroxycarboxylic acids or hydrogen peroxide, catalyzed by carboxylic acids, at temperatures from 20° C. to 90° C., wherein the molar ratio of hydrogen peroxide to the oxidizable double bond equivalents is 0.2 to 1.5 and the molar ratio of the catalytically active carboxylic acid to the oxidizable double bond equivalents is 0.2 to 1.1,
b) converting the epoxide groups formed to alcohol groups in situ by raising the temperature to 90 to 130° C., if necessary after addition of further polyols,
c) freeing the products produced in a) and b) from volatile constituents in vacuo at temperatures of 60 to 130° C. and
d) alkoxylating the products obtained in a) to c) with alkylene oxides to form polyether polyols.

The process according to the invention is even more advantageous if a further 100 to 1000 ppm of an inorganic mineral acid are additionally added in step b).

If less than one equivalent of catalytically active carboxylic acids is used in step a) per hydrogen peroxide equivalent used, it is advantageous if in addition one or more further carboxylic acids are also used in step b) until a maximum ratio of carboxylic acid equivalents to hydrogen peroxide equivalents of 1.2 is achieved. This addition of further carboxylic acid is independent of whether or not 100 to 1000 ppm of an inorganic mineral acid are added.

The process is advantageously characterized in that the unmodified unsaturated fatty acid triglyceride can be converted to the desired products by epoxidation, ring opening and alkoxylation in a single reaction vessel.

In the process according to the invention, by-products which are formed in the meantime can very easily be removed in vacuo if necessary, such that no specific purification is required for subsequent synthesis steps, which can be performed immediately afterwards.

The process according to the invention is described in detail as follows:
a), b) The triglyceride is oxidized with a peroxycarboxylic acid or a mixture of carboxylic acid and hydrogen peroxide, with which the peroxycarboxylic acid acting as oxidizing agent is formed in situ and regenerated.

Peroxyformic acid, peroxyacetic acid, trifluoroperoxyacetic acid, perbenzoic acid, 3,5-dinitroperbenzoic acid and m-chloroperbenzoic acid, for example, can be used as the peroxycarboxylic acid. Peroxyacetic acid or peroxyformic acid is preferably used. The cited acids are used in quantities of 20 to 100 mol %, based on the double bond equivalents of the triglyceride to be oxidized. To this end the triglyceride is prepared without solvent or in solution and the peroxycarboxylic acid is slowly added at 20° C. to 90° C., preferably at 35° C. to 50° C. When the addition is complete, the reaction mixture is heated for 4 to 20 hours at 90° C. to 130° C. The process is preferably performed without solvent. To support the ring-opening reaction, catalytic amounts of an inorganic mineral acid and/or alcohols can optionally also be added to the reaction mixture. The reaction process described ensures that the oxiranes that are formed as intermediates by ring opening are converted directly and completely into hydroxyl groups.

In a preferred embodiment of the epoxidation/ring-opening reaction, the peroxycarboxylic acid is produced in situ by reacting the carboxylic acid with hydrogen peroxide and re-formed during oxidation of the triglyceride. The hydrogen peroxide can be metered into the prepared mixture of triglyceride and carboxylic acid or alternatively it can be added to the triglyceride in portions at the same time as the carboxylic acid. With the latter mode of operation, exothermic reactions occurring during the oxidation reaction are more easily controlled. Formic acid or acetic acid is preferably used as the carboxylic acid. Other carboxylic acids are likewise effective here, however; thus, oxidation can be catalyzed with glycolic acid, for example. During ring opening of the oxiranes, unbound glycolic acid radicals can easily be removed by thermal treatment.

0.2 to 1.1 carboxylic acid equivalents are used per double bond equivalent to be oxidized. Depending on the desired degree of oxidation, the ratio of hydrogen peroxide equivalents to oxidizable double bond equivalents is 0.2 to 1.5. The hydrogen peroxide can be used as a solution in water with varying hydrogen peroxide contents. Solutions having a hydrogen peroxide content of 30% to 50% are preferred. The reagents are added slowly to the triglyceride at 35° C. to 80° C. Following the addition and on completion of the epoxidation reaction, the reaction mixture is heated for 4 to 20 hours to 90° C. to 130° C. This process too is preferably performed without solvent. To support the ring-opening reaction, on completion of epoxidation catalytic amounts (100 to 1000 ppm) of an inorganic mineral acid and/or alcohols can optionally be added to the reaction mixture. Surprisingly, not only do catalytic amounts of the inorganic mineral acid accelerate the ring-opening reaction of the epoxides in the anticipated manner, but in addition the products are characterized by a lower acid value, which means that firstly they are more highly defined in structural terms and secondly they can more easily undergo the alkylene oxide addition reaction d). If hypostoichiometric amounts, in other words markedly less than one equivalent of the catalytically active carboxylic acid per hydrogen peroxide equivalent, are used, in the absence of other nucleophiles the reaction substantially stops at the epoxidation stage. The ring-opening reaction of the epoxides can be completed by the addition of an adequate amount of another carboxylic acid and further heating to 90° C. to 130° C. This mode of operation allows additional functionalities to be introduced into the hydroxyl group-modified triglyceride. For example, reactive double bonds can be incorporated by using ethylene-unsaturated carboxylic acids such as acrylic acid or methacrylic acid. The use of hydroxyl group-containing carboxylic acids such as, for example, the various isomers of hydroxybenzoic acid, hydroxymethylbenzoic acid, dihydroxybenzoic acid, trihydroxybenzoic acid, hydroxyphenylacetic acid, hydroxyphthalic acid, hydroxynaphthoic acid, mandelic acid, 2-hydroxybutyric acid, 3-hydroxybutyric acid, 3-hydroxypropionic acid, tropic acid, ricinoleic acid and glyceric acid opens up access to triglyceride derivatives having higher OH values and functionalities, which are preferably used in the production of semi-rigid or rigid polyurethane foams or microcellular or compact polyurethane materials.

All triglycerides whose acid radicals are completely or partially unsaturated are suitable as substrates for the reaction sequence described in a) and b). Cottonseed oil, groundnut oil, coconut oil, linseed oil, palm kernel oil, olive oil, maize oil, palm oil, castor oil, rapeseed oil, soybean oil, sunflower seed oil, herring oil, sardine oil and tallow are cited by way of example. Refined soybean oil and rapeseed oil are preferred. Phosphoric acid is preferably used as the inorganic mineral acid which can optionally be added in a catalytic amount. Alcohols which, as mentioned above, can likewise optionally be added to the reaction mixture to support the ring-opening reaction are, for example, methanol, ethanol, ethanediol, 1-propanol, 2-propanol, 1,2- and 1,3-propanediol, 1- and 2-butanol, the isomers of butanediol, pentanol, hexanol, heptanol, octanol and decanol. Monools or monool-started alkylene oxide addition products (monofunctional polyetherols) are preferably used in the molecular weight range from 32 to 1000 g/mol.
c) Minor constituents such as water, excess carboxylic acid and optionally excess low-molecular-weight alcohol can be separated off by evacuation at 60 to 130° C. The OH value of the intermediates obtained in this way can, as already mentioned above, be adjusted by the amount of oxidizing agent used and/or by incorporating hydroxyl group-containing carboxylic acids during the ring-opening reaction. In the process according to the invention the OH value of the intermediates obtained is in the range from 50 mg KOH/g to 600 mg KOH/g.
   After addition of a catalyst and incorporation of alkylene oxides, hydroxyl group-modified triglycerides obtainable by the process described in a), b) and c) and its variants can undergo alkoxylation reactions directly and with no further processing. This reaction is preferably performed directly in the same reaction vessel. In this way the materials can very easily be modified further with regard to their OH value and their compatibility with conventional polyether polyols. Suitable alkylene oxides are, for example, ethylene oxide, propylene oxide, 1,2- or 2,3-butylene oxide and styrene oxide. Propylene oxide and ethylene oxide are preferably used, either alone or as mixtures. The polyaddition reaction can be catalyzed both by Lewis acids such as BF₃O(Et)₂ or B(C₆F₅)₃ and by bases such as alkali or alkaline-earth metal hydroxides, and by so-called double metal cyanide complex catalysts (DMC complex catalysts). The last group is preferred, since the ester bonds of the starter compounds obtainable by the processes described above are subject to no disruptive secondary reactions under the conditions of DMC catalysis. The suitability of DMC catalysts is completely surprising, however, particularly against the background of the teaching of U.S. Pat. No. 5,099,075, since the effective elimination and deactivation of DMC catalyst residues with small amounts of hydrogen peroxide at elevated temperatures are described therein.

DMC complex catalysts that are suitable for the process according to the invention are known in principle. DMC catalysts have found commercial interest above all for the production of polyether polyols by polyaddition of alkylene oxides on starter compounds displaying active hydrogen atoms, as described in U.S. Pat. Nos. 3,404,109, 3,829,505, 3,941,849 and 5,158,922, as the use of DMC catalysts brings about a reduction in the proportion of monofunctional polyethers having terminal double bonds, so-called monools, in comparison to the conventional production of polyether polyols using alkali catalysts. Furthermore, base-sensitive fictional groups such as carbonyl functions for example or the aforementioned ester groups come through DMC-catalyzed alkylene oxide addition reactions undamaged. Improved DMC catalysts, such as are described as in U.S. Pat. No. 5,470,813, EP-A 700 949, EP-A 743 093, EP-A 761 708, WO-A 97/40086, WO-A 98/16310 and WO-A 00/47649, also have an unusually high activity and allow polyether polyol production at very low catalyst concentrations of 25 ppm or less, so that separation of the catalyst from the finished product is no longer necessary. The highly active DMC catalysts described in EP-A 700 949, which in addition to a double metal cyanide compound such as zinc hexacyanocobaltate(III) and an organic complex ligand such as tert-butanol also contain a polyether having a number-average molecular weight of over 500 g/mol, are a typical example.

Before the start of the alkylene oxide addition, other compounds capable of the addition of alkylene oxides, with the aid of which for example the functionality and/or hydrophilic character of the end product can be adjusted, can also be added to the triglyceride modified by steps a), b) and c). Such starter compounds preferably have functionalities of 2 to 8. Examples are propylene glycol, ethylene glycol, diethylene glycol, 1,2-, 1,3-, 1,4-butanediol, hexanediol, pentanediol, 3-methyl-1,5-pentanediol, 1,12-dodecanediol, glycerol, trimethylol propane, triethanolamine, pentaerythritol, sorbitol, sucrose, hydroquinone, catechol, resorcinol, bisphenol F, bisphenol A, 1,3,5-trihydroxybenzene, methylol group-containing condensates of formaldehyde and phenol or melamine or urea, and Mannich bases. Examples of optionally added monoamines or polyamines are ethylene diamine, 1,3-diaminopropane, 1,4-diaminobutane, hexamethylene diamine, ethanolamine, diethanolamine, diethylene triamine, aniline, piperazine, the isomers of toluene diamine and the isomers of (diaminodiphenyl)methane. Amino group-free co-starters are preferably added. The co-starters can also already have been pre-extended in a preceding step by alkylene oxide addition, to liquefy them for example or to increase their compatibility with the reaction mixture. Suitable additional starter compounds are also poly(ester) polyols, which can be produced for example from organic dicarboxylic acids having 2 to 12 carbon atoms and polyhydric alcohols, preferably diols, having 2 to 12 carbon atoms, preferably 2 to 6 carbon atoms. Examples of possible dicarboxylic acids include: succinic acid, glutaric acid, adipic acid, suberic acid, azelaic acid, sebacic acid, decane dicarboxylic acid, dodecane dicarboxylic acid, maleic acid, fumaric acid, phthalic acid, isophthalic acid and terephthalic acid. The dicarboxylic acids can be used both individually and mixed with one another. Instead of the free dicarboxylic acids, the corresponding dicarboxylic acid derivatives can also be used, such as e.g. dicarboxylic acid monoesters and/or diesters of alcohols having 1 to 4 carbon atoms or dicarboxylic acid anhydrides. Dicarboxylic acid mixtures comprising succinic, glutaric and adipic acid, in ratios of for example 20 to 35/40 to 60/20 to 36 parts by weight, and in particular adipic acid, are preferably used. Examples of dihydric and polyhydric alcohols are ethanediol, diethylene glycol, 1,2- or 1,3-propanediol, dipropylene glycol, methyl-1,3-propanediol, 1,4-butanediol, 1,5-pentanediol, 3-methyl-1,5-pentanediol, 1,6-hexanediol, neopentylglycol, 1,10-decanediol, 1,12-dodecanediol, glycerol, trimethylolpropane and pentaerythritol. 1,2-Ethanediol, diethylene glycol, 1,4-butanediol, 1,6-hexanediol, glycerol, trimethylolpropane or mixtures of at least two of the cited diols, in particular mixtures of ethanediol, 1,4-butanediol and 1,6-hexanediol, glycerol and/or trimethylol propane, are preferably used. Polyester polyols obtained from lactones, e.g. ε-caprolactone, or hydroxycarboxylic acids, e.g. hydroxyhexanoic acid and hydroxyacetic acid, can also be used.

In order to produce such poly(ester) polyols, the organic, aromatic or aliphatic polycarboxylic acids and/or derivatives and polyhydric alcohols can undergo polycondensation free from catalysts or in the presence of esterification catalysts, conveniently in an atmosphere of inert gases, such as nitrogen, helium or argon, and also in the melt at temperatures of 150° C. to 300° C., preferably 180° C. to 230° C., optionally under reduced pressure, until the desired acid value and OH value are obtained. The acid value is advantageously less than 10 mg KOH/g, preferably less than 2.5 mg KOH/g.

According to a preferred production process, the esterification mixture undergoes polycondensation at the aforementioned temperatures under normal pressure until an acid value of 80 to 30, preferably 40 to 30 is obtained, and then under a pressure of less than 500 mbar, preferably 1 to 150 mbar. Suitable esterification catalysts include, for example, iron, cadmium, cobalt, lead, zinc, antimony, magnesium, titanium and tin catalysts in the form of metals, metal oxides or metal salts. The polycondensation of aromatic or aliphatic carboxylic acids with polyhydric alcohols can also take place in the liquid phase, however, in the presence of diluents and/or entraining agents, such as e.g. benzene, toluene, xylene or chlorobenzene, to distil off the condensation water azeotropically.

The ratio of dicarboxylic acid (derivative) and polyhydric alcohol to be chosen in order to obtain a desired OH value, functionality and viscosity and the alcohol functionalities to be chosen can be determined by the person skilled in the art by simple means.

Suitable co-starters are also poly(carbonates) displaying hydroxyl groups. Suitable examples of poly(carbonates) displaying hydroxyl groups are those of the type known per se which can be produced for example by reacting diols, such as 1,2-propanediol, 1,4-butanediol, 1,6-hexanediol, diethylene glycol, triethylene glycol, tetraethylene glycol, oligo-tetramethylene glycol and/or oligo-hexamethylene glycol with diaryl carbonates and/or dialkyl carbonates, e.g. diphenyl carbonate, dimethyl carbonate and α-ω-bischloroformates or phosgene.

The addition of alkylene oxides to the aforementioned oxidation products of triglycerides and optionally added co-starters can be continued until the OH value is as low as required. The OH value range from 5 mg KOH/g to 200 mg KOH/g is preferred. If various alkylene oxides are used, they can be added either as a blend or one at a time. With the latter addition method, the poly(ether) chains bonded to the oxidation products of the triglycerides have block structures. Pure ethylene oxide or mixtures of propylene oxide and ethylene oxide having a high proportion of ethylene oxide are preferably added as the terminal block, such that the poly(ether) chains bonded to the modified triglycerides and the optionally added co-starter compounds display 40 to 100% of primary OH terminal groups.

In accordance with the process according to the invention, the oxidized triglycerides and optionally other co-starters used as starters for the alkylene oxide addition reaction are already present in the reactor, but the starter or the mixture of starters or parts thereof can also be added to the reactor continuously during the reaction together with the alkylene oxide(s). With the latter mode of operation, an addition product comprising starter and alkylene oxide is conventionally already present in the reactor. This can be the product to be produced, but it is also possible to use an alkylene oxide addition product produced on the basis of a different starter. The addition of the reagents is followed by a post-reaction phase, during which the consumption of alkylene oxide is generally quantified by monitoring the pressure. When constant pressure is reached, the product can be removed, optionally after a curing phase.

It is also possible to remove reaction product from the reactor continuously, in which case, in addition to alkylene oxide and oxidized triglyceride and optionally other co-starters to be used, catalyst must also be metered in continuously. The various process variants for the production of polyethers by the alkylene oxide addition processes with DMC complex catalysis are described in the aforementioned prior art and in WO-A 97/29146 and WO-A 98/03571.

The DMC-catalyzed polyaddition of alkylene oxides generally takes place at temperatures from 20° C. to 200° C., preferably in the range from 40° C. to 180° C., particularly preferably at temperatures from 50° C. to 150° C. The reaction can be performed under total pressures of 0.0001 bar to 20 bar. The polyaddition can be performed without solvent or in an inert, organic solvent such as toluene and/or THF. The amount of solvent is conventionally 10 wt. % to 30 wt. %, based on the amount of end product to be produced.

The catalyst concentration is chosen such that under the cited reaction conditions, good control of the polyaddition reaction is possible. The catalyst concentration is generally in the range from 0.0005 wt. % to 1 wt. %, preferably in the range from 0.001 wt. % to 0.1 wt. %, particularly preferably in the range from 0.001 to 0.05 wt. %, based on the amount of end product to be produced.

Age resistors such as e.g. antioxidants can optionally be added to the reaction products obtained according to steps a) to d).

The polyether polyols based on oxidized triglycerides obtainable by the process according to the invention can be reacted alone or optionally mixed with other isocyanate-reactive components with organic polyisocyanates, optionally in the presence of blowing agents, in the presence of catalysts and optionally with further additives such as cell stabilizers, and used in this way as a component in the production of solid or foamed polyurethane materials.

Examples for the production of the polyols according to the invention based on unsaturated triglycerides:

### EXAMPLES

Catalyst used for the Alkylene Oxide Addition (DMC Catalyst):

Double metal cyanide catalyst, containing zinc hexacyanocobaltate, tert-butanol and polypropylene glycol, having a number-average molecular weight of 1000 g/mol; described in EP-A 700 949

IRGANOX 1076:

### Octadecyl-3-(3,5-di-tert-butyl-4-hydroxyphenyl)propionate, an antioxidant

### Example 1

604 g of soybean oil (Aldrich) were mixed with 76.4 g of formic acid at room temperature under inert gas and heated to 40° C. with stirring. 157.2 g of hydrogen peroxide (30% solution in water) were added over 55 min with stirring, whilst the temperature was held at 40° C. to 50° C. On completion of the addition of hydrogen peroxide, the batch was heated for 1 hour to 70° C. and then for 8.5 hours to 110° C. The oxirane oxygen content was 0.01%. After removing water and unreacted formic acid by distillation, the product was heated for 3 hours at 110° C. in vacuo. Yield: 665 g.

The intermediate obtained in this way had the following properties:
OH value: 171 mg KOH/g
Acid value: 10.9 mg KOH/g
Viscosity at 25° C.: 1240 mPas

132 mg of DMC catalyst were added to 500 g of the intermediate in a 10 liter laboratory autoclave. After the reaction mixture had been cured for ½ hour at 130° C. whilst passing through nitrogen and stirring at 450 rpm, the catalyst was activated at 130° C. by the addition of 50 g of propylene oxide (initial pressure: 0.1 bar, maximum pressure: 1.4 bar). The activation of the catalyst could be detected by a sudden drop in pressure in the autoclave. A further 678.6 g of propylene oxide were fed to the autoclave at 130° C. and 450 rpm over a period of 5.5 hours. After a post-reaction time of 30 min at 130° C. the alkylene oxide addition product was cured for 30 min in vacuo at 130° C. After cooling to 80° C., 614 mg of IRGANOX 1076 were added. Yield: 1224 g.

The end product obtained in this way had the following properties:
OH value: 70.4 mg KOH/g
Acid value: 18 ppm KOH
Viscosity at 25° C.: 822 mPas

### Example 2

152 g of formic acid were added dropwise to 600.2 g of soybean oil (Aldrich) at room temperature under inert gas. The mixture was heated to 40° C. with stirring. 312.5 g of hydrogen peroxide (30% solution in water) were added over 1.5 hours with stirring, whilst the temperature was held at 40° C. to 50° C. On completion of the addition of hydrogen peroxide, the batch was heated to 70° C. and once the exothermic phase had died down (temperature rise to 100° C.) it was held for 4 hours at reflux temperature (104° C.). The oxirane oxygen content was 0.01%. After removing water and unreacted formic acid by distillation, the product was heated for 3 hours at 110° C. in vacuo. Yield: 665 g.

The intermediate obtained in this way had the following properties:
OH value: 226.3 mg KOH/g
Acid value: 8.5 mg KOH/g

Viscosity at 25° C.: 21710 mPas

161 mg of DMC catalyst were added to 500 g of the intermediate in a 10 liter laboratory autoclave. After the reaction mixture had been cured for ½ hour at 130° C. whilst passing through nitrogen and stirring at 450 rpm, the catalyst was activated at 130° C. by the addition of 50 g of propylene oxide (initial pressure: 0.1 bar, maximum pressure: 1.8 bar). The activation of the catalyst could be detected by a sudden drop in pressure in the autoclave. A further 1235.7 g of propylene oxide were fed to the autoclave at 130° C. and 450 rpm over a period of 5.5 hours. After a post-reaction time of 3 hours at 130° C. the alkylene oxide addition product was cured for 30 min at 130° C. After cooling to 80° C., 893 mg of IRGANOX 1076 were added. Yield: 1775 g.

The end product obtained in this way had the following properties:
OH value: 63.5 mg KOH/g
Acid value: 32 ppm KOH
Viscosity at 25° C.: 5950 mPas

### Example 3

127 g of formic acid were added to 1005.6 g of soybean oil (Rübelmann) at room temperature under inert gas. The mixture was heated to 40° C. with stirring. 262.0 g of hydrogen peroxide (30% solution in water) were added over 1.25 hours with stirring, whilst the temperature was held at 40 to 50° C. On completion of the addition of hydrogen peroxide, the batch was heated for 1 hour to 70° C. and then held for 7 hours at 106° C. (reflux). The oxirane oxygen content was 0.05%. After removing water and unreacted formic acid by distillation, the product was heated for 3 hours at 110° C. in vacuo. Yield: 1028 g.

The intermediate obtained in this way had the following properties:
OH value: 157.2 mg KOH/g
Acid value: 7.25 mg KOH/g
Viscosity at 25° C.: 1170 mPas

268 mg of DMC catalyst were added to 971.2 g of the intermediate in a 10 liter laboratory autoclave. After the reaction mixture had been cured for ½ hour at 130° C. whilst passing through nitrogen and stirring at 450 rpm, the catalyst was activated at 130° C. by the addition of 100 g of propylene oxide (initial pressure: 0.1 bar, maximum pressure: 1.2 bar). The activation of the catalyst could be detected by a sudden drop in pressure in the autoclave. A further 1351 g of propylene oxide mixed with 180 g of ethylene oxide were fed to the autoclave at 130° C. and 450 rpm over a period of 2.5 hours. After a post-reaction time of 40 min at 130° C. the alkylene oxide addition product was cured for 30 min at 130° C. After cooling to 80° C., 1327 mg of IRGANOX 1076 were added. Yield: 2589 g.

The end product obtained in this way had the following properties:
OH value: 58.8 mg KOH/g
Acid value: not detectable
Viscosity at 25° C.: 920 mPas

### Example 4

152.1 g of formic acid were added to 600.6 g of soybean oil (Rübelmann) at room temperature under inert gas. The mixture was heated to 40° C. with stirring. 312.5 g of hydrogen peroxide (30% solution in water) were added over 1.5 hours with stirring, whilst the temperature was held at 40° C. to 50° C. On completion of the addition of hydrogen peroxide, the batch was heated to 70° C. and once the exothermic phase had died down (temperature rise to 100° C.) it was held for 4 hours at reflux temperature (103° C.). The oxirane oxygen content was less than 0.01%. After removing water and unreacted formic acid by distillation, the product was heated for 3 hours at 110° C. in vacuo. Yield: 631 g.

The intermediate obtained in this way had the following properties:
OH value: 212.5 mg KOH/g
Acid value: 6.95 mg KOH/g
Viscosity at 25° C.: 22800 mPas

207 mg of DMC catalyst were added to 555.6 g of the intermediate in a 10 liter laboratory autoclave. After the reaction mixture had been cured for ½ hour at 130° C. whilst passing through nitrogen and stirring at 450 rpm, the catalyst was activated at 130° C. by the addition of 55 g of propylene oxide (initial pressure: 0.1 bar, maximum pressure: 1.8 bar). The activation of the catalyst could be detected by a sudden drop in pressure in the autoclave. A further 1265.1 g of propylene oxide mixed with 157.7 g of ethylene oxide were fed to the autoclave at 130° C. and 450 rpm over a period of 3 hours. After a post-reaction time of 23 min at 130° C. the alkylene oxide addition product was cured for 30 min at 130° C. After cooling to 80° C., 1017 mg of IRGANOX 1076 were added. Yield: 2023 g.

The end product obtained in this way had the following properties:
OH value: 58.4 mg KOH/g
Viscosity at 25° C.: 2280 mPas

### Example 5

198.7 g of acetic acid and 188.0 g of hydrogen peroxide (as a 50% solution in water) were each added in 7 equal portions to 600 g of soybean oil (Rübelmann) under nitrogen at 70° C. over a period of 6 hours and 15 min. Each hydrogen peroxide portion was added immediately after an acetic acid portion. After the final portion had been added, the reaction mixture was left to react for 1 hour at 70° C. and then heated for 8 hours to reflux temperature (102° C. to 106° C.). The oxirane oxygen content was 0.05%. After removing water and unreacted acetic acid by distillation, the product was heated for 3 hours at 110° C. in vacuo. Yield: 663 g.

The intermediate obtained in this way had the following properties:
OH value: 201.5 mg KOH/g
Acid value: 16.2 mg KOH/g
Viscosity at 25° C.: 7920 mPas

100 mg of DMC catalyst were added to 315.6 g of the intermediate in a 2 liter laboratory autoclave. After the reaction mixture had been cured for ½ hour at 130° C. whilst passing through nitrogen and stirring at 800 rpm, the catalyst was activated at 130° C. by the addition of 31.5 g of propylene oxide (initial pressure: 0.1 bar, maximum pressure: 2.1 bar). The activation of the catalyst could be detected by a sudden drop in pressure in the autoclave. A further 701.5 g of propylene oxide were fed to the autoclave at 130° C. and 800 rpm over a period of 4 hours and 50 min. After a post-reaction time of 40 min at 130° C. the alkylene oxide addition product was cured for 30 min at 130° C. After cooling to 80° C., 553 mg of IRGANOX 1076 were added. Yield: 1037 g.

The end product obtained in this way had the following properties:
OH value: 60.9 mg KOH/g
Acid value: 20.1 ppm KOH
Viscosity at 25° C.: 3552 mPas

### Example 6

330.2 g of acetic acid and 312.3 g of hydrogen peroxide (as a 50% solution in water) were each added in 7 equal portions to 2000 g of soybean oil (Rübelmann) under nitrogen at 70° C. over a period of 6 hours. Each hydrogen peroxide portion was added immediately after an acetic acid portion. After the final portion had been added, the mixture was left to react for 1 hour at 70° C. 1.32 g of phosphoric acid were added and the reaction mixture then heated for 7 hours to reflux temperature (about 105° C.). The oxirane oxygen content was 0.06%. After removing water and unreacted acetic acid by distillation, the product was heated for 3 hours at 110° C. in vacuo (1 mbar). Yield: 2125 g.

The intermediate obtained in this way had the following properties:
OH value: 150.0 mg KOH/g
Acid value: 8.5 mg KOH/g
Viscosity at 25° C.: 870 mPas

203 mg of DMC catalyst were added to 826.4 g of the intermediate in a 10 liter laboratory autoclave. After the reaction mixture had been cured for ½ hour at 130° C. whilst passing through nitrogen and stirring at 800 rpm, the catalyst was activated at 130° C. by the addition of a mixture of 73.5 g of propylene oxide and 9.0 g of ethylene oxide (initial pressure: 0.1 bar, maximum pressure: 2.7 bar). The activation of the catalyst could be detected by a sudden drop in pressure in the autoclave. A further 118.4 g of ethylene oxide and 971.4 g of propylene oxide were fed to the autoclave at 130° C. and 800 rpm over a period of 1 hour and 23 min. After a post-reaction time of 36 min at 130° C. the alkylene oxide addition product was cured for 30 min at 130° C. After cooling to 80° C., 1091 mg of IRGANOX 1076 were added. Yield: 1990 g.

The end product obtained in this way had the following properties:
OH value: 62 mg KOH/g
Acid value: 19.3 ppm KOH
Viscosity at 25° C.: 909 mPas

In a comparative example, which was performed in the first stage without the addition of phosphoric acid, the mixture had to be heated for 18 hours to 105° C. to obtain <0.1% oxirane oxygen in the intermediate. The intermediate had an acid value of 21.1 mg KOH/g. The subsequent addition of alkylene oxides could not take place as the DMC catalyst could not be activated.

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. A process for the production of polyols based on natural oils comprising:
a) oxidizing unsaturated triglycerides with peroxycarboxylic acids or hydrogen peroxide, catalyzed by carboxylic acids, at temperatures from about 20° C. to about 90° C., wherein the molar ratio of hydrogen peroxide to the oxidizable double bond equivalents is from about 0.2 to about 1.5 and the molar ratio of catalytically active carboxylic acid to oxidizable double bond equivalents is from about 0.2 to about 1.1,
b) converting the epoxide groups formed to alcohol groups in situ by raising the temperature to from about 90 to about 130° C., if necessary after addition of further polyols,
c) freeing products produced in a) and b) from volatile constituents in vacuo at temperatures of from about 60 to about 130° C. and
d) alkoxylating the products obtained in a) to c) with alkylene oxides to form polyether polyols.
e) The process according to claim 1, wherein a further about 100 to about 1000 ppm of an inorganic mineral acid are additionally added in step b).

2. The process according to claim 1, wherein the peroxycarboxylic acid is selected from the group consisting of peroxyformic acid, peroxyacetic acid, trifluoroperoxyacetic acid, perbenzoic acid, 3,5-dinitroperbenzoic acid and m-chloroperbenzoic acid,

3. The process according to claim 1, wherein additionally one or more further carboxylic acids can also be used in step b) until a maximum ratio of carboxylic acid groups to the hydrogen peroxide equivalents of about 1.2 is achieved.

4. The process according to claim 1, wherein reaction steps a) to d) are performed in a single reaction vessel.

5. The process according to claim 1, wherein the unsaturated triglyceride is selected from the group consisting of cottonseed oil, groundnut oil, coconut oil, linseed oil, palm kernel oil, olive oil, maize oil, palm oil, castor oil, rapeseed oil, soybean oil, sunflower seed oil, herring oil, sardine oil and tallow.

6. The process according to claim 1, wherein the unsaturated triglyceride is selected from soybean oil and rapeseed oil.

7. The process according to claim 1, wherein the alkylene oxide is selected from the group consisting of ethylene oxide, propylene oxide, 1,2- or 2,3-butylene oxide, styrene oxide and mixtures thereof.
